(19) United States
(12) Patent Application Publication
(10) Pub. No.: US 2023/0157689 A1
(43) Pub. Date: May 25, 2023

(54) ARTICULATING PITUITARY RONGEUR FOR USE WITH A CANNULA

(71) Applicant: Life Spine, Inc., Huntley, IL (US)

(72) Inventor: Daniel P. Predick, Wheat Ridge, CO (US)

(73) Assignee: Life Spine, Inc., Huntley, IL (US)

(21) Appl. No.: 17/981,675

(22) Filed: Nov. 7, 2022

Related U.S. Application Data
(60) Provisional application No. 63/282,444, filed on Nov. 23, 2021.

(51) Int. Cl. A61B 17/072; A61B 17/28

(52) U.S. Cl. CPC A61B 17/07207; A61B 17/28; A61B 2017/00398

(57) ABSTRACT

A pituitary rongeur medical instrument has jaws that can controllably articulate radially from a longitudinal axis of a shaft assembly of the pituitary rongeur and beyond a perimeter of a cannula during use. The pituitary rongeur is characterized by a handle, a shaft assembly defining a proximal end extending from the handle, a distal end, and a longitudinal axis, with the jaws pivotally connected to the distal end of the shaft assembly for articulating radial movement of the jaws relative to the longitudinal axis of the shaft assembly, a jaw controller associated with the shaft assembly and configured to controllably close and open the jaws, and an articulation controller associated with the shaft assembly and configured to controllably articulate the jaws. The handle is formed by a handgrip portion configured to receive a palm of a user and a lever portion with a finger loop configured to receive fingers of the user.

### CROSS-REFERENCE TO RELATED APPLICATIONS

This U.S. non-provisional patent application claims the benefit of and/or priority under 35 U.S.C. § 119(e) to U.S. provisional patent application Ser. No. 63/282,444 filed Nov. 23, 2021 titled “Articulating Pituitary Rongeur,” the entire contents of which is specifically incorporated herein by reference.

### FIELD OF THE INVENTION

The present invention relates to instruments for spine procedures and, more particularly, to pituitary rongeurs.

### BACKGROUND OF THE INVENTION

Many people contend with spine issues due to age, disease, trauma, congenital, and acquired complications and conditions. While some spine issues can be alleviated without surgery, other spine issues necessitate surgery. Spine surgery may entail removing a vertebral disc or disc material from the vertebral disc space between vertebrae of the spine. One such medical instrument used in spine surgery is known as a pituitary rongeur. A pituitary rongeur is used to obtain and remove vertebral discs and/or vertebral tissue (vertebral disc material) from a spine. A pituitary rongeur has jaws at a distal end of a shaft assembly that is manipulated by a handle. The jaws obtain and remove portions of the vertebral disc and/or tissue. The jaws may or may not have teeth.

Spine procedures such as minimally invasive spine surgery, micro spine surgery, and other spine procedures often use pituitary rongeurs. These types of procedures have advantages over conventional procedures such as reduction of trauma, better post operative recovery, less pain, and the like through use of specialized surgical instruments that are introduced into the body via a separate cannula which is inserted into the body through a small incision and positioned accordingly. When a pituitary rongeur is used, it is inserted into the previously positioned cannula until the jaws of the pituitary rongeur extend beyond the distal end of the inserted cannula. The jaws of the pituitary rongeur are then manipulated to obtain vertebral disc material (e.g. vertebral discs, disc material and/or other vertebral tissue) for removal from the body. The pituitary rongeur is then removed from the cannula where the acquired disc material and/or tissue can be removed from the jaw(s). Other instruments may be used for tissue extraction.

Heretofore, jaws of pituitary rongeurs have been fixed in co-axial orientation relative to a longitudinal axis of the pituitary rongeur. This limits reach of the jaws and thus the ability of the jaws to obtain more vertebral disc material from a single position of the cannula.

In view of the above, it would be advantageous to have a medical instrument fashioned as a pituitary rongeur for removing vertebral discs, disc tissue, and/or other vertebral tissue via a cannula, such as in a minimally invasive, micro invasive, or similar spine procedure, having an operating head with jaws that can extend beyond a perimeter and longitudinal axis of the cannula. It would furthermore be advantageous to have a pituitary rongeur medical instrument for use with a cannula during a minimally invasive, micro invasive or other spine procedure that may provide better access to the vertebral disc space, greater removal of vertebral discs/disc material, greater degree of sweep, and extension beyond the longitudinal axis of the cannula and of the elongated pituitary rongeur medical instrument. Other advantages are known but not enumerated.

The present pituitary rongeur medical instrument addresses the above and more.

### SUMMARY OF THE INVENTION

A medical instrument is formed as a pituitary rongeur having jaws that articulate radially relative to a longitudinal axis of a shaft assembly of the pituitary rongeur for obtaining and removing vertebral discs, disc tissue, and other vertebral tissue (vertebral material) from vertebral disc spaces of a spine via a cannula, particularly, but not necessarily, during minimally invasive surgery, micro surgery, or similar spine procedure.

In one form, a handle trigger actuates a push rod (jaws control rod) that forces a wedge surface under a superior (upper) jaw portion of the jaws forcing its closure (for obtaining vertebral material) onto an inferior (lower) jaw portion of the jaws. The wedge surface feature of the push rod has a surface that facilitates articulation of superior jaw while the superior jaw is in line with the longitudinal axis of the instrument and in any position up to 90 degrees of jaw articulation. Jaws articulation is actuated via a pull rod (articulation control rod) that has a hinged interface with the inferior jaw portion. The jaw portions are forced into an open position via an integrated leaf spring. Operation of the pituitary rongeur permits insertion down an access tube (cannula, endoscope or the like), then, once articulated, can gather disc material from outside a perimeter (radially from) of the access tube.

The pituitary rongeur has a handle, a shaft assembly defining a proximal end extending from the handle, a distal end, and a longitudinal axis, upper and lower jaw portions forming a jaw or jaws pivotally connected to the distal end of the shaft assembly for articulating radial movement relative to the longitudinal axis of the shaft assembly, a first (jaws) controller associated with the shaft assembly and configured to controllably close and open the jaws, and a second (articulation) controller associated with the shaft assembly and configured to controllably articulate the jaws.

In one form, the handle is formed by a handgrip portion configured to receive a palm of a user and a lever portion with a finger loop configured to receive one or more fingers of the user. A double leaf spring is situated on and between the handgrip portion and the lever portion to provide tension between the handgrip portion and the lever portion when the handgrip portion and the lever portion are squeezed together during use, and to automatically return the lever portion to an initial state when squeezing of the handgrip portion and the lever portion has ceased. The handgrip portion may include a thumb hold.

The upper and/or lower jaw portions may or may not have teeth. Preferably, but not necessarily, the jaws are normally open.

Further aspects of the present invention will become apparent from consideration of the drawings and the following description of a form of the invention. A person skilled in the art will realize that other forms of the invention are possible and that the details of the invention can be modified in a number of respects without departing from the inventive concept. The following drawings and description are to be regarded as illustrative in nature and not restrictive.

### BRIEF DESCRIPTION OF THE DRAWINGS

The present invention and its features will be better understood by reference to the accompanying drawings, wherein:

FIG. 1 is a view of a medical instrument formed as an articulating pituitary rongeur fashioned in accordance with the present principles for removing vertebral discs and tissue from a vertebral disc space of a spine via a cannula;

FIG. 2 is an enlarged view of a shaft assembly of the articulating pituitary rongeur of FIG. 1;

FIG. 3 is an enlarged view of the distal end of the shaft assembly and articulating jaws of the articulating pituitary rongeur of FIG. 1 with the jaws in a co-a axial position relative to the longitudinal axis of the shaft assembly and in a normally open position;

FIG. 4 is another enlarged view of the distal end of the shaft assembly and articulating jaws of the articulating pituitary rongeur of FIG. 1 with the jaws in the co-axial position relative to the longitudinal axis of the shaft assembly and in the normally open position;

FIG. 5 is a further enlarged view of the distal end of the shaft assembly and articulating jaws of the articulating pituitary rongeur of FIG. 1 with the jaws in the co-axial position relative to the longitudinal axis of the shaft assembly and in the normally open position;

FIG. 6 is an enlarged view of the distal end of the shaft assembly and articulating jaws of the articulating pituitary rongeur of FIG. 1 with the jaws in a radially articulated position and in the normally open position;

FIG. 7 is another enlarged view of the distal end of the shaft assembly and articulating jaws of the articulating pituitary rongeur of FIG. 1 with the jaws in another radially articulated position and in the normally open position, the view illustrating the range of articulation of the jaws/jaw assembly relative to the longitudinal axis of the shaft assembly;

FIG. 8 is a further enlarged view of the distal end of the shaft assembly and articulating jaws of the articulating pituitary rongeur of FIG. 1 with the jaws in the radially articulated position of FIG. 7 but in a semi-closed position;

FIG. 9 is a yet further enlarged view of the distal end of the shaft assembly and articulating jaws of the articulating pituitary rongeur of FIG. 1 with the jaws in the radially articulated position of FIG. 7 but in a closed position;

FIG. 10 is an enlarged side view of the distal end of the shaft assembly and articulating jaws of the articulating pituitary rongeur of FIG. 1 taken along line 10-10 of FIG. 9 with the jaws in the radially articulated position of FIG. 7 and in the closed position;

FIG. 11 is another enlarged side view of the distal end of the shaft assembly and articulating jaws of the articulating pituitary rongeur of FIG. 1 taken along line 11-11 of FIG. 9 with the jaws in the radially articulated position of FIG. 7 and in the closed position;

FIG. 12 is an enlarged view of a distal end of a cannula with the jaws of the articulating pituitary rongeur of FIG. 1 shown emerging from the distal end of the cannula, in a co-axial position with the longitudinal axis of the shaft assembly, and in the closed position;

FIG. 13 is a further enlarged view of the distal end of the cannula with the jaws of the articulating pituitary rongeur of FIG. 1 shown emerging from the distal end of the cannula, in a co-axial position with the longitudinal axis of the shaft assembly, and in the closed position as depicted in FIG. 12;

FIG. 14 is a yet further enlarged view of the distal end of the cannula with the jaws of the articulating pituitary rongeur of FIG. 1 shown emerged from the distal end of the cannula, in a co-axial position with the longitudinal axis of the shaft assembly, and in the closed position;

FIG. 15 is a still further enlarged view of the distal end of the cannula with the jaws of the articulating pituitary rongeur of FIG. 1 shown emerged from the distal end of the cannula, in a co-axial position with the longitudinal axis of the shaft assembly, and in an open position;

FIG. 16 is a furthermore enlarged view of the distal end of the cannula with the jaws of the articulating pituitary rongeur of FIG. 1 shown emerged from the distal end of the cannula, and in an open and radially articulated position;

FIG. 17 is a yet furthermore enlarged view of the distal end of the cannula with the jaws of the articulating pituitary rongeur of FIG. 1 shown emerged from the distal end of the cannula, and in the closed and radially articulated position;

FIG. 18 is a still furthermore enlarged view of the distal end of the cannula with the jaws of the articulating pituitary rongeur of FIG. 1 shown emerged from the distal end of the cannula, in a co-axial position with the longitudinal axis of the shaft assembly, and in the closed position;

FIG. 19 is an additional enlarged view of the distal end of the cannula with the jaws of the articulating pituitary rongeur of FIG. 1 shown retracting into the distal end of the cannula, in a co-axial position with the longitudinal axis of the shaft assembly, and in the closed position;

FIG. 20 is an enlarged sectional side view of the jaws of the articulating pituitary rongeur of FIG. 1 in the closed position; and

FIG. 21 is another enlarged sectional side view of the jaws of the articulating pituitary rongeur of FIG. 1 in an open position.

For the purposes of promoting an understanding of the principles of the invention, reference will now be made to the embodiment illustrated in the drawings and specific language will be used to describe the same. It will nevertheless be understood that no limitation of the scope of the invention is thereby intended, any alterations and further modifications in the described embodiment, and any further applications of the principles of the invention as described herein are contemplated as would normally occur to one skilled in the art to which the invention relates.

### DETAILED DESCRIPTION OF THE INVENTION

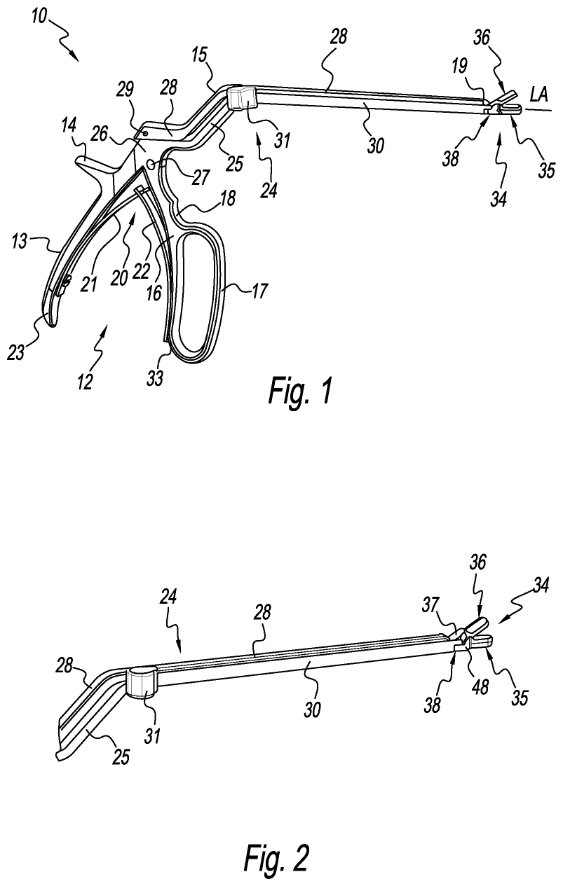

Referring to FIG. 1, there is shown a medical instrument formed as an articulating pituitary rongeur, generally designated 10, for use with an access tube such as, but not limited to, a cannula, endoscope or the like (collectively, cannula), in acquiring vertebral discs, vertebral tissue, and/or other tissue or material (not shown), from a vertebral disc space (not shown) between adjacent vertebra (not shown) of a spine (not shown) during a surgical spine procedure, preferably, but not necessarily, in minimally invasive or micro invasive spine surgery. The articulating pituitary rongeur 10 is made from one or more surgical grade materials. The articulating pituitary rongeur 10 has a handle assembly 12, a shaft assembly 24 defining a proximal end 15 extending from the handle assembly 12, a distal end 19, and a longitudinal axis LA. A jaw assembly or simply, jaws 34 is pivotally connected to the distal end 19 of the shaft assembly 24 for articulating radial movement of the jaws 34 relative to the longitudinal axis LA of the shaft assembly 24, and includes a first (jaw) control shaft 28 associated with the shaft assembly 24 and configured to control opening and closing of an upper jaw portion 36 of the jaws 34 relative to a lower jaw portion 35 of the jaws 34, and a second (articulation) controller 30 associated with the shaft assembly 24 and configured to control articulation of the jaws 34, the nomenclature first and second being arbitrary here and throughout unless specified otherwise.

The handle assembly 12 is characterized by a first handle portion 13 and a second handle portion 16 pivotally connected at pivot pin 27 to an upper handle portion 26 of the first handle portion 13. The first handle portion 13 is preferably, but not necessarily, formed to accept a palm (not shown) of a hand (not shown) of a user (not shown) and preferably, but not necessarily, includes a flange 14 extending generally outward from the first handle portion 13 and formed to receive a thumb (not shown) of the user (not shown) as a thumb rest. The second handle portion 16 has a loop 17 formed at its distal end 33 that is styled to receive the three lower fingers—i.e. middle, ring, and pinkie (not shown), of the hand (not shown) of the user (not shown). The loop 17 may accommodate other fingers. The second handle portion 16 further includes a curved notch 18 axially above the finger loop 17 that is formed to receive an index or other finger (not shown) of the user's hand (not shown). Other configurations may be used and are contemplated.

The handle assembly 12 is configured to be normally in an open position as depicted in FIG. 1 with the jaws 34 also normally in an open position as also depicted in FIG. 1. Situated between the first handle portion 13 and the second handle portion 17 is a spring assembly 20 fashioned as a double leaf spring having a first leaf spring 21 connected to a second leaf spring 22. Other types of springs mat be used. The first leaf spring 21 is connected to an inside of the first handle portion 13 proximal to its distal end 23, while the second leaf spring 22 is connected to an inside of the second handle portion 16 proximal to its distal end 33. Application of pressure to (e.g. squeezing of) the second handle portion 17 towards the first handle portion 13 against tension of the double leaf spring 20 pivots the second handle portion 17 to close the jaws 34. Release of pressure (squeezing) against the second handle portion 17 causes the spring assembly 20 to pivot the second handle portion 17 away from the first handle portion.

Referring additionally to FIG. 2, the shaft assembly 24 includes a main or stationary shaft 25 that extends from the upper handle portion 26 to the jaws 34. The first control shaft 28 likewise extends from the upper handle portion 26 to the jaws 34. The first control shaft 28 is connected to the main shaft 25 by a sliding attachment such that the first control shaft 28 can translate along the main shaft 25. The first control shaft 28 is labeled twice in FIGS. 1 and 2 to illustrate its extension length along the main shaft 25 such that axial movement along the longitudinal axis LA of the shaft assembly 24 provides control of the closing and opening of the upper jaw portion 36 of the jaws 34 relative to the lower jaw portion 35 of the jaws 34, and may be considered a jaw control shaft. The first control shaft 28 is connected to the second handle portion 16 via a pin 29 creating a lever/lever action there-between, whereby squeezing of the second handle portion 16 towards the first handle portion 13 pivots the second handle portion 16 on the pivot pin 27 such that the jaw control shaft 28 axially moves toward the jaws 34, while release of squeezing of the second handle portion 16 allows the spring assembly 20 to reverse pivot the second handle portion 16 relative to the first handle portion 13 which, in turn, axially moves the jaw control shaft 28 away from the jaws 34. Axial movement of the jaw control shaft 28 toward the jaws 34, closes the upper jaw portion 36 onto the lower jaw portion 35, while axial movement of the jaw control shaft 28 away from the jaws 34 allows the upper jaw portion 36 to bias itself into an open position relative to the lower jaw portion assembly 35.

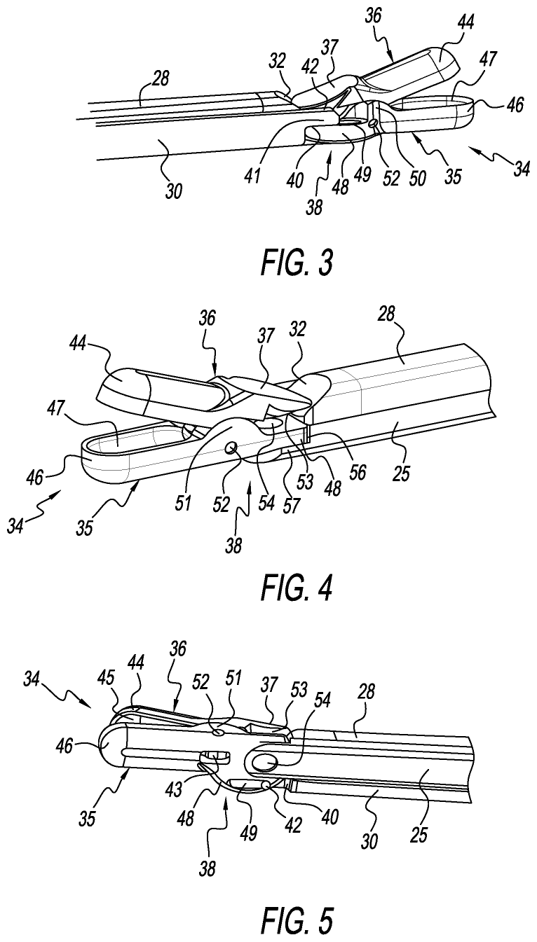

Referring to FIGS. 3-11, 20, and 21, the lower jaw portion 35 of the jaws 34 will now be described. The lower jaw portion 35 includes a base 48 with a lower jaw 46 having a lower jaw cavity 47. A first lower jaw assembly boss 51 extends from a first lateral side of the base 48, and a second lower jaw assembly boss 50 extends from a second lateral side of the base 48. As best seen in FIGS. 5, 20, and 21, the lower jaw portion 35 is pivotally connected to the main shaft 25 by a pivot post 54. The main shaft 25 ends in a notch 56 that defines a ledge 57. The ledge 57 has a bore 55 while the base 48 also includes a bore 39. The pivot post 54 extends through the base bore 39 and the ledge bore 55 such that the base 48 pivots radially on and relative to the main shaft 25 and its longitudinal axis LA or 0°, which pivots the lower jaw portion 35, and consequently the upper jaw portion 36 since the upper jaw portion 36 is connected to the lower jaw portion 35 as described below. The base 48 and thus the lower and upper jaw portions 35, 36 (jaws/jaw assembly) can swivel (articulate) generally laterally +90° to −90° relative to the longitudinal axis LA of 0° (see, e.g. FIG. 7). The jaws/jaw assembly may only swivel (articulate) to one lateral side and thus only from 0° to 90° and back.

Articulation of the jaws 34 is controlled by a second (articulation) control shaft 30 that is connected to a lateral side of the main shaft 25 so as to slide along the lateral side of the main shaft 25. Connection may be accomplished e.g., via a dovetail configuration between the main shaft 25 and the articulation control shaft 30. Other configurations may be used. A flange 31 is provided at a distal end of the articulation control shaft 30 that is configured to allow a user use a finger or thumb to control longitudinal movement of the articulation control shaft 30, which in turn articulates the jaws/jaw assembly 34 to swing radially to the left or right relative to distal end of the shaft assembly 24 and its longitudinal axis LA.

As best seen in FIGS. 3 and 5, the distal end of the articulation control shaft 30 has a notch 40 that defines a flange 41. A control pin 42 extends downward from the flange 41 and into a slot 49 of the base 48. Longitudinal movement of the articulation control shaft 30 in one axial direction on the shaft assembly articulates the jaws/jaw assembly 34 in a first radial (lateral) direction (from 0° to either +90° or −90°), while longitudinal movement of the articulation control shaft 30 in the opposite axial direction on the shaft assembly articulates the jaws/jaw assembly 34 in a second radial (lateral) direction (from 0° to either −90° or +90°) opposite the first radial direction. The nomenclature + and − being arbitrary.

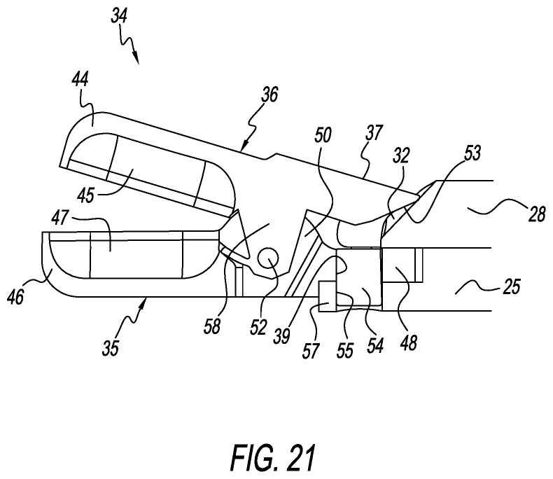

The upper jaw portion 36 of the jaws 34 will now be described. The upper jaw portion 36 includes an upper jaw 44 having an upper jaw cavity 45. The upper jaw cavity 45 and the lower jaw cavity 47 receive and hold vertebral disc tissue. As best seen in FIGS. 20 and 21, the upper jaw portion 36 also includes a second upper jaw portion boss 58 that is pivotally coupled to the first lower jaw portion boss 50 by a pivot pin 52. While not seen in the Figures, the upper jaw portion 36 further includes a first upper jar portion boss 59 that is pivotally coupled to the first lower jaw portion boss 51 via the pivot pin 52. The upper jaw portion 36 further includes a flat-topped end or flange 37 situated opposite the upper jaw 44 having a lower surface 53. The lower surface 53 is configured to contact a slanted distal end 32 of the jaw control shaft 28. The upper jaw portion 36 is biased to be normally in an open position as depicted in at least FIGS. 3-6.

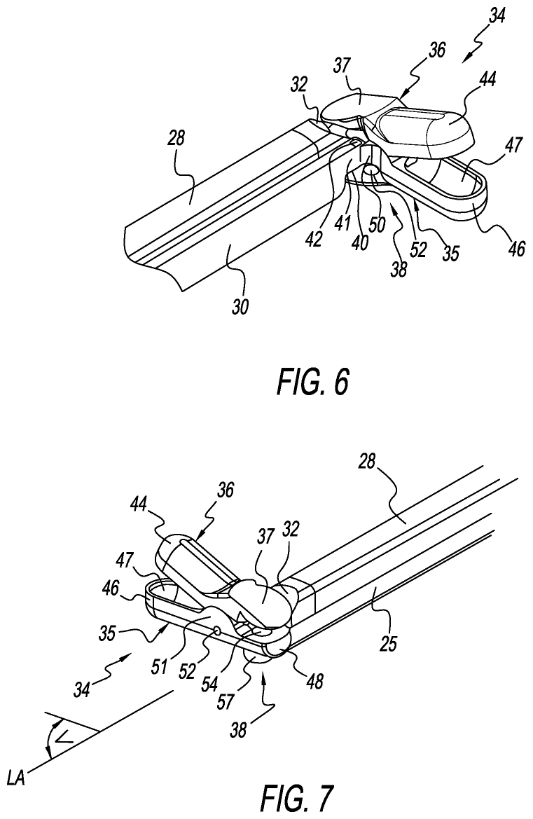

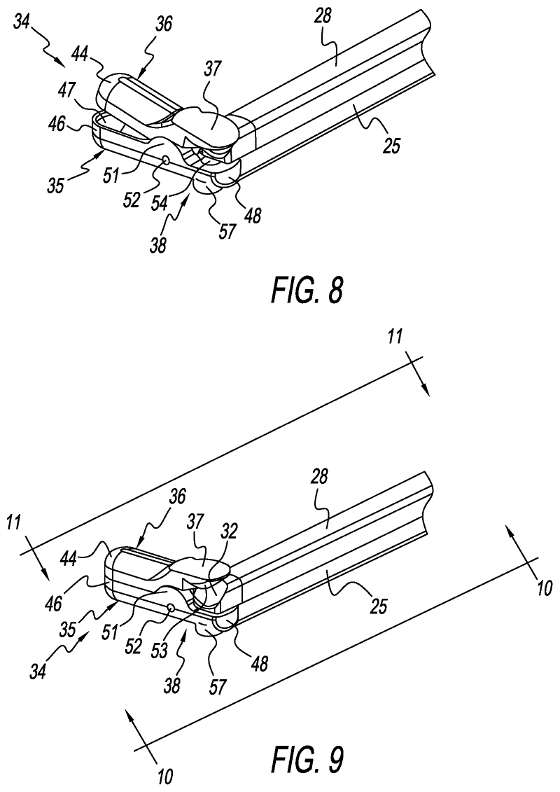

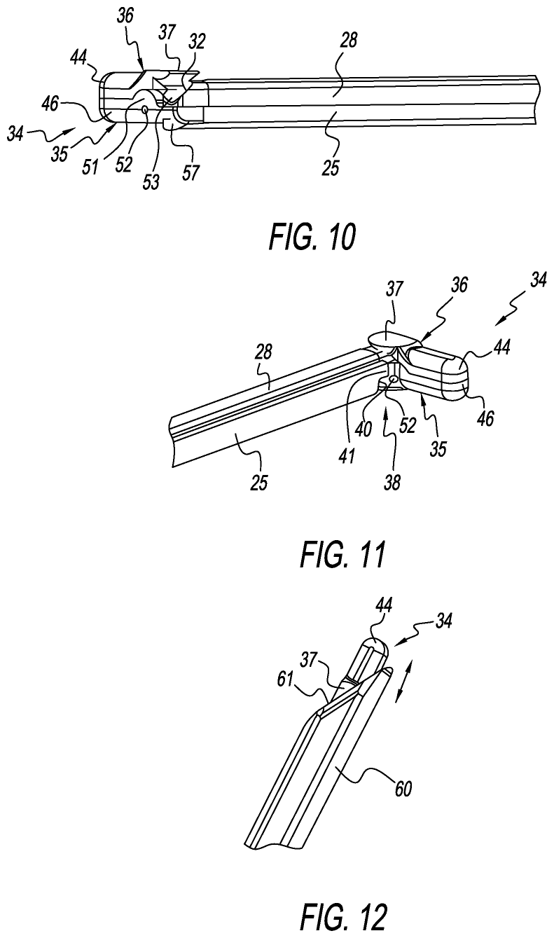

FIGS. 3-5 depict the jaws 34 in the biased normally open position. The jaw control shaft 28 is in an axially rearward position and the articulation control shaft 30 in a neutral position wherein the jaws 34 are co-axial with the longitudinal axis LA of the shaft assembly 24. FIGS. 6 and 7 depict two lateral views of the jaws 34 in the biased normally open position with the jaw control shaft 28 in the axially rearward position but articulated into a generally +90° position by the articulation control shaft 30 being pulled axially rearward (i.e. toward the handle 12). FIG. 8 depicts the lateral view of FIG. 7 but with the upper jaw portion 36 partially closed through axial movement of the jaw control shaft 28 towards the jaw assembly wherein the slanted distal end 32 of the jaw control shaft 28 moves under the flat-topped end or flange 37 of the upper jaw portion 36 to contact the underside 53 to push the upper jaw 44 towards the lower jaw 46. FIG. 9 depicts the upper jaw portion 36 fully closed through further axial movement of the jaw control shaft 28 towards the jaws 34 wherein the slanted distal end 32 of the jaw control shaft 28 further moves under the flat-topped end or flange 37 of the upper jaw portion 36 to contact the underside 53 to further push the upper jaw 44 towards and onto the lower jaw 46. FIG. 10 depicts the view of FIG. 9 taken along line 10-10 thereof. FIG. 11 depicts the view of FIG. 9 taken along line 11-11 thereof.

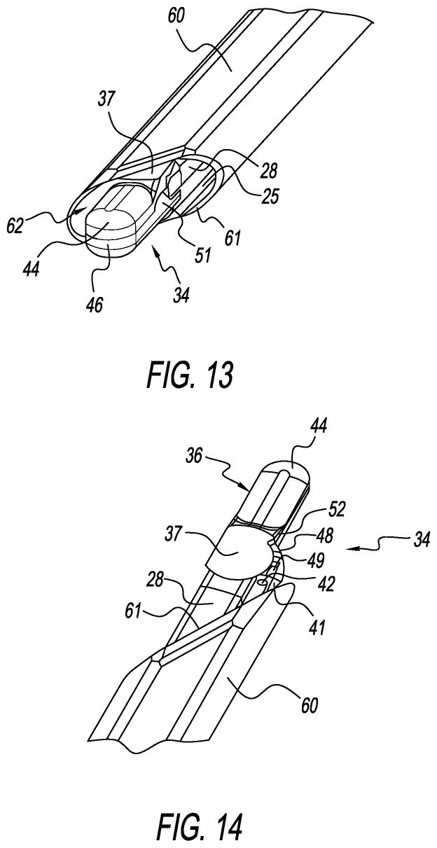

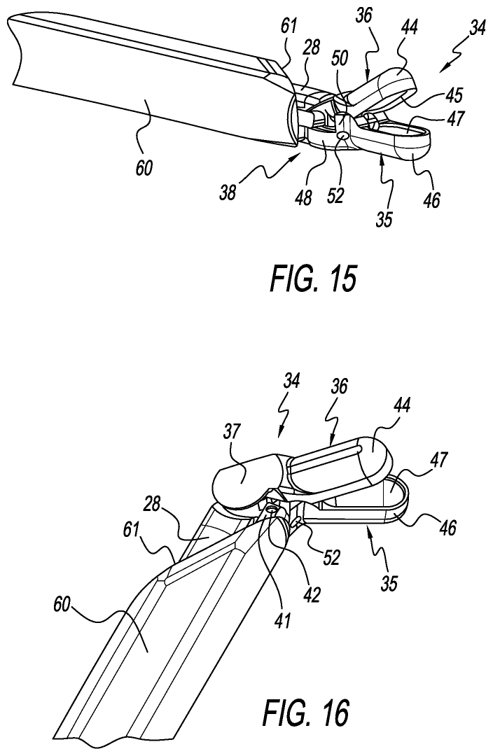

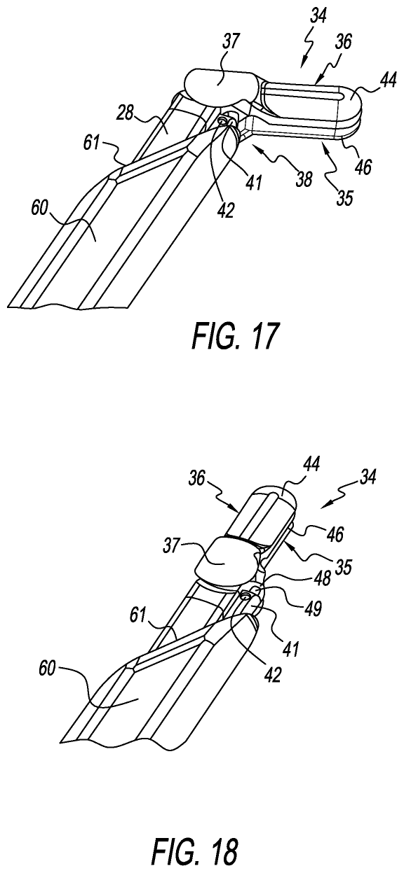

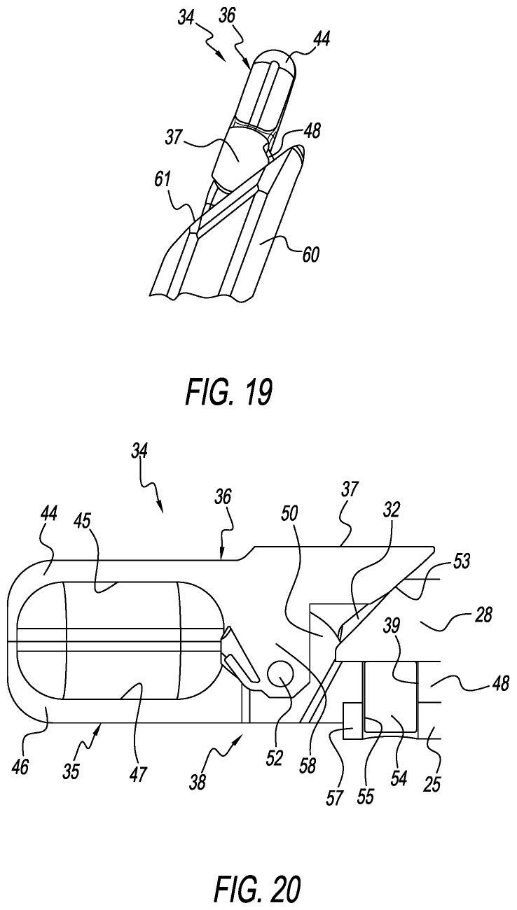

The articulating pituitary rongeur 10 is particularly, but not necessarily, intended and configured for minimally or micro invasive spine surgery or similar spine procedure, and thus the use of a cannula (access tube or tubes). FIGS. 12-19 depict one such use, inferring if not describing, others along with the present figures. FIGS. 12-19 also show a sequence or method of use of the articulating pituitary rongeur 10 with an exemplary cannula 60. The cannula 60 has a central bore 62 and a slanted distal end 61. In the sequence or method of FIGS. 12-19, the jaws/jaw assembly 34 is shown emerging from the end 61 of the cannula 60 wherein the jaws 34, being constrained by the cannula 60 to be in the closed position, opens (FIGS. 12-15). The sequence or method further shows the jaws 34 articulating to a radial position relative to the longitudinal axis LA of the shaft assembly 24 (FIG. 16), then closing the upper jaw/jaw portion 44 onto the lower jaw/jaw portion 46 (FIG. 17). Thereafter, while still closed (after obtaining disc tissue) the jaw assembly 34 retracts back into the cannula 60 for removal for the obtained disc material (FIGS. 18, 19). The double-headed arrow of FIG. 12 signifies the longitudinally axial movement of (emerging from and retracting into) the pituitary rongeur 10 within the cannula 60.

While the invention has been illustrated and described in what is presently considered to be the most practical and preferred embodiment, it is to be understood that the invention is not to be limited to the disclosed embodiments, but on the contrary, is intended to cover various modifications and equivalent arrangements included within the spirit and scope of the appended claims, which scope is to be accorded the broadest interpretation so as to encompass all such modifications and equivalent structures as permitted under the law. Accordingly, the scope of the present invention should be considered in terms of the following claims and is understood not to be limited to the details of structure and operation shown and described in the specification and drawings.

It should be understood that while the use of the word preferable, preferably, or preferred in the description above indicates that feature so described may be more desirable, it nonetheless may not be necessary and any embodiment lacking the same may be contemplated as within the scope of the invention, that scope being defined by the claims that follow. In reading the claims it is intended that when words such as “a,” “an,” “at least one” and “at least a portion” are used, there is no intention to limit the claim to only one item unless specifically stated to the contrary in the claim. Unless specifically stated to the contrary in the claim, the language “at least one of X, Y, and Z” should be interpreted as including both the conjunctive and disjunctive forms. Specifically, the language “at least one of X, Y, and Z” is intended to encompass the following permutations of X, Y, and Z: X alone; Y alone; Z alone; X and Y; X and Z; Y and Z; and X, Y, and Z. Further, when the language “at least a portion” and/or “a portion” is used the item may include a portion and/or the entire item unless specifically stated to the contrary.

1. A medical instrument for obtaining vertebral disc material from a spine during a surgical spine procedure utilizing an access tube, the medical instrument comprising:
a handle defining a hand-operated lever with a first elongated portion having a thumb hold and a second elongated portion having a finger grip;
a shaft assembly defining a proximal shaft assembly end, a distal shaft assembly end opposite the proximal shaft assembly end, and a longitudinal axis, the proximal shaft assembly end connected to the handle;
jaws at the distal shaft assembly end and configured to articulate laterally relative to the longitudinal axis of the shaft assembly, the jaws in a normally open position;
a first controller associated with the shaft assembly and defining a proximal first controller end and a distal first controller end opposite the proximal first controller end, the proximal first controller end connected to the hand-operated lever and the distal first controller end in communication with the jaws to controllably close the jaws via the hand-operated lever; and
a second controller associated with the shaft assembly and defining a proximal second controller end and a distal second controller end opposite the proximal second controller end and configured to controllably articulate the jaws laterally relative to the longitudinal axis of the shaft assembly and beyond a perimeter of the shaft assembly.

2. The medical instrument of claim 1, wherein the hand-operated lever includes a pivot pin extending through the first elongated portion and the second elongated portion such that the second elongated portion pivots with respect to the first elongated portion, with the proximal first controller end of the first controller slidingly connected to the second elongated portion whereby pivoting motion of the second elongated portion translates the first controller to controllably close the jaws.

3. The medical instrument of claim 2, further comprising a leaf spring situated on and between the first elongated portion and the second elongated portion to provide tension between the first elongated portion and the second elongated portion when the first elongated portion and the second elongated portion are squeezed together during use, and to automatically return the hand-operated lever to an initial state when squeezing of the first elongated portion and the second elongated portion has ceased.

4. The medical instrument of claim 3, wherein the leaf spring comprises a double leaf spring.

5. The medical instrument of claim 2, wherein:
the jaws comprise an upper jaw portion pivotally attached to a lower jaw portion;
the distal first controller end has a first wedge surface; and
the upper jaw portion has a second wedge surface situated opposite to the first wedge surface, wherein the first and second wedge surfaces abut to cause pivoting of the upper jaw portion relative the lower jaw portion.

6. The medical instrument of claim 5, wherein the upper jaw portion has teeth.

7. The medical instrument of claim 6, wherein the lower jaw portion has teeth.

8. The medical instrument of claim 5, wherein the jaws can articulate up to 90° relative to the longitudinal axis of the shaft assembly.

9. An articulating pituitary rongeur for obtaining and removing vertebral disc tissue from a vertebral disc space of a spine during a surgical spine procedure using a cannula, the articulating pituitary rongeur comprising:
a handle defining a hand-operated lever with a first elongated portion having a thumb hold and a second elongated portion having a finger grip;
a shaft assembly defining a proximal shaft assembly end, a distal shaft assembly end opposite the proximal shaft assembly end, and a longitudinal axis, the proximal shaft assembly end connected to the handle;
a jaw assembly at the distal shaft assembly end and configured to articulate laterally relative to the longitudinal axis of the shaft assembly, the jaw assembly in a normally open position;
a first jaw controller situated on the shaft assembly and defining a proximal first jaw controller end and a distal first jaw controller end opposite the proximal first jaw controller end, the proximal first jaw controller end connected to the hand-operated lever and the distal first jaw controller end in communication with the jaw assembly to controllably close the jaw assembly via the hand-operated lever; and
a second jaw controller associated with the shaft assembly and defining a proximal second jaw controller end and a distal second jaw controller end opposite the proximal second jaw controller end and configured to controllably articulate the jaw assembly laterally relative to the longitudinal axis of the shaft assembly and beyond a perimeter of the shaft assembly.

10. The articulating pituitary rongeur of claim 9, wherein the hand-operated lever includes a pivot pin extending through the first elongated portion and the second elongated portion such that the second elongated portion pivots with respect to the first elongated portion, with the proximal first jaw controller end of the first jaw controller slidingly connected to the second elongated portion whereby pivoting motion of the second elongated portion translates the first controller to controllably close the jaw assembly.

11. The articulating pituitary rongeur of claim 10, further comprising a leaf spring situated on and between the first elongated portion and the second elongated portion to provide tension between the first elongated portion and the second elongated portion when the first elongated portion and the second elongated portion are squeezed together during use, and to automatically return the hand-operated lever to an initial state when squeezing of the first elongated portion and the second elongated portion has ceased.

12. The articulating pituitary rongeur of claim 11, wherein the leaf spring comprises a double leaf spring.

13. The articulating pituitary rongeur of claim 10, wherein:
the jaw assembly comprises an upper jaw portion pivotally attached to a lower jaw portion;
the distal first controller end has a first wedge surface; and
the upper jaw portion has a second wedge surface situated opposite to the first wedge surface, wherein the first and second wedge surfaces abut to cause pivoting of the upper jaw portion relative the lower jaw portion.

14. The articulating pituitary rongeur of claim 13, wherein the upper jaw portion has teeth.

15. The articulating pituitary rongeur of claim 14, wherein the lower jaw portion has teeth.

16. The articulating pituitary rongeur of claim 15, wherein the jaw assembly can articulate up to 90° relative to the longitudinal axis of the shaft assembly.

17. A method of obtaining and removing vertebral discs and tissue from a vertebral disc space of a spine during a surgical spine procedure using an access tube, the method comprising:
inserting an access tube in a patient proximate to a vertebral disc space;
introducing a medical instrument into the access tube, the medical instrument having:
a handle defining a hand-operated lever with a first elongated portion having a thumb hold and a second elongated portion having a finger grip;
a shaft assembly defining a proximal shaft assembly end, a distal shaft assembly end opposite the proximal shaft assembly end, and a longitudinal axis, the proximal shaft assembly end connected to the handle;
a jaw assembly at the distal shaft assembly end and configured to articulate laterally relative to the longitudinal axis of the shaft assembly, the jaw assembly in a normally open position;
a first jaw controller situated on the shaft assembly and defining a proximal first jaw controller end and a distal first jaw controller end opposite the proximal first jaw controller end, the proximal first jaw controller end connected to the hand-operated lever and the distal first jaw controller end in communication with the jaw assembly to controllably close the jaw assembly via the hand-operated lever; and
a second jaw controller associated with the shaft assembly and defining a proximal second jaw controller end and a distal second jaw controller end opposite the proximal second jaw controller end and configured to controllably articulate the jaw assembly laterally relative to the longitudinal axis of the shaft assembly and beyond a perimeter of the shaft assembly;
manipulating the medical instrument to obtain vertebral disc material in the jaw assembly; and
removing the medical instrument from the access tube with the obtained vertebral disc material in the jaw assembly.

18. The method of claim 17, wherein:
the hand-operated lever of the medical instrument includes a pivot pin extending through the first elongated portion and the second elongated portion such that the second elongated portion pivots with respect to the first elongated portion, with the proximal first jaw controller end of the first jaw controller slidingly connected to the second elongated portion whereby pivoting motion of the second elongated portion translates the first controller to controllably close the jaw assembly; and
the medical instrument further comprises a double leaf spring situated on and between the first elongated portion and the second elongated portion to provide tension between the first elongated portion and the second elongated portion when the first elongated portion and the second elongated portion are squeezed together during use, and to automatically return the hand-operated lever to an initial state when squeezing of the first elongated portion and the second elongated portion has ceased.

19. The method of claim 18, wherein:
the jaw assembly of the medical instrument comprises an upper jaw portion pivotally attached to a lower jaw portion;
the distal first controller end of the medical instrument has a first wedge surface;
the upper jaw portion has a second wedge surface situated opposite to the first wedge surface, wherein the first and second wedge surfaces abut to cause pivoting of the upper jaw portion relative the lower jaw portion; and
wherein at least one of the upper and lower jaw portions has teeth.

20. The method of claim 19, wherein the jaw assembly of the medical instrument can articulate up to 90° relative to the longitudinal axis of the shaft assembly.
